(19) United States
(12) Patent Application Publication
(10) Pub. No.: US 2007/0276083 A1
(43) Pub. Date: Nov. 29, 2007

(54) LUSTER MATERIAL-CONTAINING RESIN COMPOSITION

(75) Inventors: Kenichi HIGASHI, Brussels (BE); Mitsuyoshi Shimano, Ichihara-shi (JP)

Correspondence Address: FITCH, EVEN, TABIN & FLANNERY, P. O. BOX 18415, WASHINGTON, DC, 20036, US

(73) Assignees: Sumitomo Chemical Company, Limited, Tokyo (JP); Uchihamakasei Corp., Toyoto-shi (JP); Sumika Color Co., Ltd., Osaka (JP)

(21) Appl. No.: 11/748,516

(22) Filed: May 15, 2007

(30) Foreign Application Priority Data
Nov. 16, 2005 (JP) P2005-331257

(51) Int. Cl. A61K 9/16

(52) U.S. Cl. 524/543

(57) ABSTRACT

A resin composition containing (A) 100 parts by weight of a thermoplastic resin, (B) 1 to 70 parts by weight of a fibrous material having an average fiber length of 0.1 to 20 mm, and (C) 0.1 to 10 parts by weight of a luster material, which provides a molded article with good metallic appearance.

### FIELD OF THE INVENTION

The present invention relates a luster material-containing resin composition, which can provide a molded article having metallic appearance with no weld line

### BACKGROUND ART

Hitherto, molded articles of thermoplastic resins are used as exterior parts, interior parts, parts in engine compartments of automobiles. To impart metallic appearance or pearlescent appearance to such resin molded articles, in particular, exterior and interior parts, colorants such as metal powder, pearlescent pigments, etc. are added to the resins.

For example, JP-A-08-239549 discloses a resin composition comprising a crystalline ethylene-propylene block copolymer having an ethylene content of 2 to 15% by weight and a Rockwell hardness of at least 85, an ethylene-α-olefin copolymer having an ethylene content of 80 to 95% by weight, an inorganic filler and a colorant, as a resin composition which has gloss, metallic appearance and large color depth, and provides a molded article with excellent mechanical properties.

However, when the resin composition of JP-A-08-239549 is used to produce exterior parts, interior parts, parts in engine compartments of automobiles, these parts tend to have poor appearance due to the formation of weld lines or nonuniform orientation of metal powder, etc., since they have complicated product shapes with openings, ribs, bosses, the change of wall thickness, and so on. Therefore, it has been desired to avoid the formation of weld lines and to uniformly orient the metal powder, etc.

### SUMMARY OF THE INVENTION

An object of the present invention is to provide a luster material-containing resin composition which can provide a molded article having excellent metallic appearance with no weld line.

To achieve the above object, the present invention provides a resin composition comprising (A) 100 parts by weight of a thermoplastic resin, (B) 1 to 70 parts by weight of a fibrous material having an average fiber length of 0.1 to 20 mm, and (C) 0.1 to 10 parts by weight of a luster material.

### BRIEF DESCRIPTION OF THE DRAWINGS

FIG. 1 is a perspective view of a molded article produced in the Examples, in which the unit of sizes is

FIG. 2 is a perspective view of a test piece produced in the Examples, in which the unit of sizes is “mm”.

### DETAILED DESCRIPTION OF THE INVENTION

The thermoplastic resin (A) in the composition of the present invention may be any one of commonly used thermoplastic resins. Examples of the thermoplastic resin (A) include polypropylene resins, polyethylene resins, polyamide resins, ABS (acrylonitrile-butadiene-styrene) resins, polystyrene, polyphenylene ether resins, and blends and alloys of two or more of these resins. Among them, polypropylene resins are preferable.

Specific examples of the polypropylene resins include propylene homopolymers, propylene-α-olefin random copolymers, propylene-ethylene block copolymers, mixtures thereof, etc. Among the polypropylene resins, propylene-ethylene block copolymers are preferable from the viewpoint of the weight-lightening and impact resistance of the molded articles made of the resin composition of the present invention.

When thermoplastic resin (A) is a propylene-α-olefin random copolymer, examples of the α-olefin include ethylene and α-olefins having 4 to 8 carbon atoms such as butene-1, hexene-1, octene-1, etc. The content of repeating units derived from the α-olefin(s) in the propylene-α-olefin random copolymer is preferably from 1 to 49% by weight.

A propylene-ethylene block copolymer used as the thermoplastic resin (A) may be a copolymer comprising at least one crystalline propylene-homopolymer block formed by the homopolymerization of propylene and at least one copolymer block formed by the copolymerization of ethylene and propylene.

The content of the crystalline propylene-homopolymer block in the propylene-ethylene block copolymer is preferably from 60 to 95% by weight, while the content of the copolymer block is preferably from 40 to 5% by weight, each based on the whole weight of the propylene-ethylene block copolymer, from the viewpoint of the weight-lightening and impact resistance of the molded articles made of the resin composition of the present invention. The content of repeating units derived from ethylene in the copolymer block is preferably from 10 to 60% by weight.

The fibrous material (B) in the composition of the present invention has an average fiber length of 0.1 to 20 mm. The fibrous material may be inorganic fiber or organic fiber, and preferably inorganic fiber. Specific examples of the inorganic fiber include glass fiber, carbon fiber, etc., and specific examples of the organic fiber include polyester fiber, aramid fiber, etc.

The average fiber length of the fibrous material (B) is preferably from 0.3 to 15 mm, more preferably 0.5 to 10 mm. When the fibrous material (B) has an average fiber length in such a range, the molded article produced from the resin composition of the present invention has fewer weld lines and thus better appearance.

The content of the fibrous material (B) in the resin composition of the present invention is usually from 1 to 70 parts by weight per 100 parts by weight of the thermoplastic resin (A), and it is preferably from 2 to 60 parts by weight, more preferably 3 to 50 parts by weight, further preferably from 5 to 45 parts by weight, from the viewpoint of the strength, heat resistance, dimensional stability and weight-lightening of the molded article produced from the resin composition.

The luster material (C) preferably comprises metal particles, for example, particles of aluminum, copper, a copper alloy, gold, silver, etc. The luster material (C) may be in the form of spheres or flakes having a ratio of a major axis to a minor axis of at least 5 and a thickness of 0.5 to 10 μm. The luster material (C) is preferably aluminum particles, more preferably flake-form aluminum particles, since a metallic molded article having better color depth can be obtained.

The average particle size of the luster material (C) is preferably from 1 to 200 μm, more preferably from 5 to 180 μm, further preferably from 90 to 180 μm, from the viewpoint of the improvement of the luster appearance of the molded article produced from the resin composition without deteriorating the flowability or the composition, and from the viewpoint of diminishing the appearance of weld lines which are formed at meeting regions of the parts of the resin composition in the molded article. Herein, the “average particle size” of the metal particles means an “average diameter” in the case of spherical particles, or an “average major axis length” in the case of the flake-form particles.

The content of the luster material (C) in the resin composition of the present invention is from 0.1 to 10 parts by weight per 100 parts by weight of the thermoplastic resin (A). When the resin composition of the present invention contains the luster material (C) in an amount of 0.1 to 10 parts by weight, it can provide a molded article with good luster appearance, and furthermore, it has appropriate flowability so that it can easily be molded. The content of the luster material (C) is preferably from 0.1 to 5 parts by weight, more preferably 0.3 Lo 4.5 parts by weight, further preferably from 0.5 to 4 parts by weight, per 100 parts by weight of the thermoplastic resin (A).

When a luster material (C) having a large average particle size is used, its content is preferably reduced, while when a luster material (C) having a small average particle size is used, its content is preferably increased, from the viewpoint of diminishing the appearance of weld lines which are formed at meeting regions of the parts of the resin composition in the molded article. For example, when the average particle size is 90 μm, the content of the luster material (C) is preferably at least 3 parts by weight, when 100 μm, preferably at least 1.2 parts by weight, and when 120 μm, preferably at least 0.2 part by weight, since the appearance of the weld lines is particularly diminished.

In one preferred embodiment of the present invention, an average particle size X (μm) of the luster material (C) and a content Y (parts by weight) of the luster material (C) per 100 part by weight of the resin composition preferably satisfy the following relationship:
Y>(X-122)/(-11)

If the average particle size X and the content Y of the luster material (C) satisfy the above relationship, the poor appearance of molded articles caused by the weld lines and also black streaks formed in the welded regions can be avoided, when the molded articles having complicated shapes with openings, the change of wall thickness, and the like are produced using a mold having a plurality of gates.

The resin composition of the present invention may optionally contain an inorganic filler (D). The inorganic filler (D) is a filler other than the luster material (C). Examples of the other filler include talc, calcium carbonate, barium sulfate, calcium silicate, clays, magnesium carbonate, silica, etc These fillers may be used singly, or as a mixture of two or more of them. Among them, calcium carbonate is preferable since the weld appearance is particularly improved.

The content of the inorganic filler (D) is preferably from 1 to 50 parts by weight per 100 parts by weight of the thermoplastic resin (A). In view of the dimensional stability and stiffness of the molded article, the content of the inorganic filler (D) is more preferably from 3 to 50 parts by weight, still more preferably from 5 to 50 parts by weight, and most preferably from 5 to 45 parts by weight, per 100 parts by weight of the thermoplastic resin (A).

Furthermore, the resin composition of the present invention may optionally contain a thermoplastic elastomer (E). Examples of the thermoplastic elastomer (E) include ethylene-propylene copolymer elastomers, ethylene-propylene-conjugated diene copolymer elastomers, ethylene-α-olefin copolymer elastomers, styrene-butadiene-styrene block copolymer elastomers, styrene-isoprene-styrene block copolymer elastomers, styrene-ethylene-butylene-styrene block copolymer elastomers, etc. Among them, ethylene-propylene copolymer elastomers and ethylene-α-olefin copolymer elastomers are preferable.

The content of the thermoplastic elastomer (E) is preferably from 1 to 50 parts by weight per 100 parts by weight of the thermoplastic resin (A). In view of the impact strength, dimensional stability and stiffness of the molded article, the content of the thermoplastic elastomer (E) is more preferably from 5 to 50 parts by weight, still more preferably from 5 to 45 parts by weight, per 100 parts by weight of the thermoplastic resin (A).

The resin composition of the present invention may optionally contain a modified polypropylene resin (F) which comprises at least one kind of repeating units selected from the group consisting of repeating units derived from an unsaturated carboxylic acid and repeating units derived from an unsaturated carboxylic acid anhydride from the viewpoint of the prevention of agglomeration of the luster material during molding and the improvement of the appearance of the molded article.

The addition of the modified polypropylene resin (F) to the resin composition of the present invention can effectively prevent the agglomeration of the luster material in a cylinder of an injection molding machine or in a hot runner of a mold. Furthermore, the resin composition containing the modified polypropylene resin (F) can easily provide a molded article having deep metallic or pearlescent appearance and also good appearance with fewer streaks.

The amount of the modified polypropylene resin (F) to be added to the resin composition is usually from 0.05 to 5 parts by weight, preferably from 0.1 to 4 parts by weight, per 100 parts by weight of the resin composition, since the resin composition has appropriate flowability so that it can easily be molded, and it provides a molded article with excellent impact strength.

The modified polypropylene resin (F) may be prepared by, for example,

(1) a method comprising polymerizing at least one monomer selected from the group consisting of unsaturated carboxylic acids and unsaturated carboxylic acid anhydrides (hereinafter referred to as “characteristic monomer”) and a monomer other than the characteristic monomer, or

(2) a method comprising reacting the characteristic monomer with a polymer comprising a monomer other than the characteristic monomer.

The modified polypropylene resin (F) may be a random copolymer resin or a graft copolymer resin.

Preferable examples of a monomer other than the characteristic monomer include ethylene, propylene, styrene, etc. As the monomer other than the characteristic monomer, which is copolymerized by the method (1), a single sort of monomer or a mixture of two or more sorts of monomers may be used. The polymer with which the characteristic monomer is reacted may be a homopolymer of a single sort of monomer or a copolymer of two or more sorts of monomers. Hereinafter, the “repeating units derived from an unsaturated carboxylic acid” and “repeating units derived from an unsaturated carboxylic acid anhydride” are collectively referred to as “characteristic repeating units”.

Examples of the unsaturated carboxylic acid include monocarboxylic acids such as acrylic acid, methacrylic acid, etc.; and dicarboxylic acids such as maleic acid, fumaric acid, itaconic acid, etc. Examples of the unsaturated carboxylic acid anhydride include monocarboxylic acid anhydrides such as acrylic anhydride, methacrylic anhydride, etc.; and dicarboxylic acid anhydrides such as maleic anhydride, phthalic anhydride, itaconic anhydride, etc. The modified polypropylene resin (F) is preferably a modified polypropylene resin comprising at least one kind of repeating units selected from the group consisting of repeating units derived from a dicarboxylic acid and repeating units derived from a dicarboxylic acid anhydride, from the viewpoint of the prevention of the agglomeration of the luster material.

The content of the characteristic repeating units is preferably from 0.01 to 20% by weight, more preferably from 0.1 to 10% by weight, further preferably from 0.1 to 5% by weight, from the viewpoint of the effective prevention of the agglomeration of the luster material and thus the easy production of a molded article having good appearance with no streak, and also from the viewpoint that the resin composition has appropriate flowability so that it has good moldability, and it provides a molded article with excellent impact strength. When one polymer chain (or molecule) contains two or more different kinds of characteristic repeating units, the total content of the characteristic repeating units in one polymer chain is preferably from 0.01 to 20% by weight, more preferably from 0.1 to 10% by weight, further preferably from 0.1 to 5% by weight. The modified polypropylene resins may be used singly or as a mixture of two or more of them.

In some applications, a partial coating may be applied, a printed foil may be transferred, or a polyurethane foam may be bonded to the surface of the molded article made of the resin composition of the present invention. In such cases, to attain sufficient adhesion force, the resin composition of the present invention may contain at least one additive (G) selected from the group consisting of reaction products of the modified polypropylene resin (F) with low molecular weight diols, low molecular weight diamines or low molecular weight compounds having a hydroxyl group and an amino group, terpene-phenol resins and petroleum resins.

Examples of the low molecular weight diols, the low molecular weight diamines and the low molecular weight compounds having a hydroxyl group and an amino group include diols such as ethylene glycol, diethylene glycol, 1,4-butanediol, bishydroxyethoxybenzene, etc.; diamines such as ethylenediamine, 1,4-butylenediamine, cyclohexanediamine, tolylenediamine, isophoronediamine, etc.; low molecular weight compounds having a hydroxyl group and an amino group such as alkanolamines (e.g. monoethanolamine, diethanolamine, etc.). Among them, the compounds having a hydroxyl group and an amino group are preferable. The content of the additive (G) is usually from 1 to 15 parts by weight per 100 parts by weight of the resin composition of the present invention.

Preferred examples of the additive (G) include modified polypropylene prepared by reacting maleic anhydride-modified polypropylene and monoethanolamine. The modified polypropylene preferably has a molecular weight Mn of 2,000 to 20,000 from the viewpoint of the improvement of the impact strength and flowability of the resin composition. The modified polypropylene preferably has a hydroxyl value of 10 to 50 from the viewpoint of the improvement of the adhesion of the coating, printed foil or polyurethane foam to the resin composition, from the viewpoint of the prevention of the agglomeration of the modified polypropylene in the resin composition, and also from the viewpoint of the improvement of the elastic modulus and impact strength of the resin composition. Examples of the commercially available additive (G) include YUMEX® 1210H and YUMEX® 1210 (both manufactured by Sanyo Chemical Industries, Ltd.).

If necessary, the resin composition of the present invention may optionally contain a metal deactivator. Examples of the metal deactivator include benzotriazole derivatives disclosed in “NEW DEVELOPMENTS OF POLYMER ADDITIVES” (KOUBUNSHI TENKAZAI NO SHINTENKAI), p. 76-85 (published by the Nikkan Kogyo Shimbun, Ltd.) and JP-A-08-302331, compounds having at least one —CO—NH— group (e.g. oxalic acid derivatives, salicylic acid derivatives, hydrazide derivatives, hydroxybenzanilide derivatives, etc.), sulfur-containing phosphites, melamines, and so on. Among them, compounds having at least one —CO—NH— group (e.g. oxalic acid derivatives, salicylic acid derivatives and hydrazide derivatives), sulfur-containing phosphates and melamines are preferable. The molded article produced from the resin composition comprising the metal deactivator is preferably used as a part which should have heat resistance, for example, a part used in an engine compartment, since it has excellent heat resistance.

Furthermore, the resin composition of the present invention may optionally contain one or more conventional additives, if necessary. Examples of the conventional additive include neutralizing agents, antioxidants, UV-absorbers, antistatic agents, lubricants, processing aids, colorants, blowing agents, antimicrobials, organic peroxides, plasticizers, flame retardants, nucleating agents, dispersants, crosslinking agents, crosslinking aids, etc. These additives may be used singly or as a mixture of two or more of them. Among these conventional additives, antioxidants and colorants are frequently used. Examples of the antioxidants include phenol-base antioxidants and phosphor-base antioxidants, sulfur-base antioxidants. They may be used singly or as a mixture of two or more of them. The content of the additives is arbitrarily selected, and may usually be from 0.01 to 2 parts by weight per 100 parts by weight of the resin composition.

The colorant may be a conventional colorant such as an inorganic or organic pigment. Examples of the inorganic pigment include iron oxide, titanium oxide, zinc oxide, colcothar, cadmium red, cadmium yellow, ultramarine blue pigment, cobalt blue pigment, titanium yellow, lead white, red lead, lead yellow, iron blue pigment, etc. Examples of the organic pigment include quinacridone, polyazo yellow, anthraquinone yellow, polyazo red, azo lake yellow, perylene, phthalocyanine green, phthalocyanine blue, isoindolinone yellow, etc. These colorants may be used singly or as a mixture of two or more of them. The particle size and amount of the colorant may arbitrarily be selected.

The resin composition of the present invention may be prepared by a conventional method, for example, (1) a method comprising dry blending components (A), (B) and (C) and also optional other components, and then melt kneading the blended components, and (2) a method comprising charging components (A), (B) and (C) and also optional other components directly to a kneader and kneading them in the process of the production of a molded article.

In the production of the resin composition of the present invention, a master batch comprising a thermoplastic resin containing an increased amount of the luster material (C) may be used.

The resin composition of the present invention can be molded by a suitable molding method to form a molded article. The molding method may be injection molding, injection compression molding, compression molding, extrusion, blow molding, etc.

The molded article made of the resin composition of the present invention is preferably used as an automobile part such as an exterior part, an interior part, a cowling (an engine cover), a part used in an engine compartment, etc.

The present invention will be illustrated by the following examples, which do not limit the scope of the present invention in any way.

In the Examples, a following injection molding machine, a mold, molding conditions and evaluation methods were used.

(1) Injection Molding Machine, Mold and Molding Conditions

(A) Production of sample for evaluating weld appearance

Injection molding machine: FS 160 ASEN manufactured by Nissei Plastic Industrial Co., Ltd.; Mold clamping force: 160 tons

Molding temperature: 220° C.

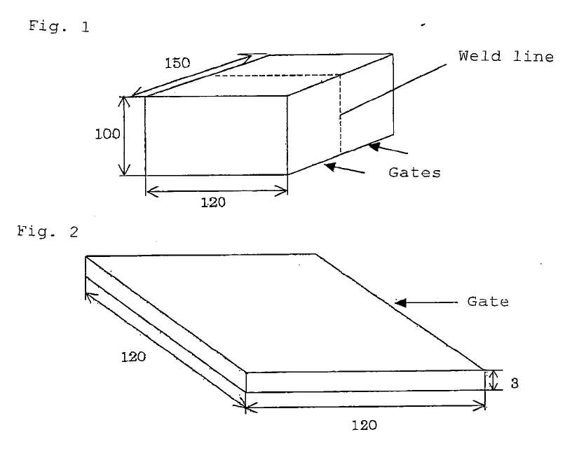

Mold: Molded article thickness: 3 mm; two point gates; Shape: see FIG. 1

Mold temperature: 40° C.

(B) Production of test piece for measuring fiber length and test piece for measuring heat resistance

Injection molding machine: J 100E manufactured by the Japan Steel Works, Ltd.; Mold clamping force: 100 tons

Molding temperature: 220° C.

Mold: Outer sizes of molded article: 120 mm×120 mm×3 mm thick; Film gate; Shape: see FIG. 2

Mold temperature: 40° C.

(2) Measurement of Average Fiber Length

A small piece (2 g) was sampled from the central region of the test piece having outer sizes of 120 mm×120 mm×3 mm thick produced under the above conditions, and heated in an oven kept at a temperature of 500° C. or higher for 3 hours or longer to ash it. Then, the ash was magnified with an optical microscope, the length of each of 100 fibers which were randomly selected was measured, and then the measured lengths of 100 fibers were averaged to obtain an average fiber length.

(3) Evaluation of Weld Appearance

Welded areas formed in a region of the molded article in a range of 100 mm to 220 mm from the gates were visually observed, and the presence of black streak-like lines was evaluated and ranked according to the following criteria: A: No black streak-like weld line observed; B: Black streak-like weld lines clearly observed.

(4) Heat Resistance Test

The molded article produced under the above conditions was heated at 150° C. for 150 hours. The color difference of the molded article before and after the heat treatment was obtained by measuring a ΔE value of the sample using a SM color computer (Model SM-5 manufactured by Suga Test Instruments Co., Ltd.). As a reference sample, the sample prior to the heat resistance test was used. The color was measured using a 45 degree diffusion type optical instrument (SM-5-CH type).

### EXAMPLE 1

As a combination of a thermoplastic resin (A) and a fibrous material (B), glass fiber-containing polypropylene (Sumitomo Noblen® GHH32 manufactured by Sumitomo Chemical Co., Ltd.; glass fiber content: 30% by weight; polypropylene content 70% by weight; MFR=3 g/10 min.) was used. Then, 100 parts by weight of glass fiber-containing polypropylene, 3.4 parts by weight of an aluminum paste (manufactured by Toyo Aluminum Kabushiki-kaisha; average particle size: 90 μm) [4.9 parts by weight per 100 parts by weight of the polypropylene resin (A)] as a luster material (C), 0.2 part by weight of ADK STAB® ZS-90 (manufactured by ADEKA CORPORATION) [0.28 part by weight per 100 parts by weight of the polypropylene resin (A)] as a metal deactivator and, as other additives, 0.1 part by weight of Sumilizer® GA80 (an antioxidant manufactured by Sumitomo Chemical Co., Ltd.), 0.1 part by weight of Ultranox® 626 (an antioxidant manufactured by GE Specialty Chemicals, Inc.) and 0.3 part by weight of Sumilizer® TPM (an antioxidant manufactured by Sumitomo Chemical Co., Ltd.) were kneaded and granulated at 230° C. with a twin-screw extruder having a diameter of 40 mm to obtain a resin composition.

The resin composition prepared in the previous step was molded to produce a box-shaped molded article as shown in FIG. 1. The molded article had silver metallic appearance. It had no black streak-like weld line.

Then, a test piece having the outer sizes of 120 mm×120 mm×3 mm thick, shown in FIG. 2, was placed in an oven kept at 150° C. After heating the test piece for 150 hour, a ΔE value was measured to evaluate color change. The result is shown in Table 1.

### COMPARATIVE EXAMPLE 1

One hundred (100) parts by weight of an ethylene-propylene block copolymer (Sumitomo Noblen® AZ 864 manufactured by Sumitomo Chemical Co., Ltd.; MFR=30 g/10 min.) as a thermoplastic resin (A), 27 parts by weight of talc (MW HS-T manufactured by Hayashi-Kasei Co., Ltd.) as an inorganic filler (D), 1.3 parts by weight of an aluminum paste (manufactured by Toyo Aluminum Kabushiki-kaisha; average particle size: 10 μm) as a luster material (C), 0.25 part by weight of ADK STAB® ZS-90 (manufactured by ADEKA CORPORATION) as a metal deactivator and, as other additives, 0.1 part by weight of Sumilizer® GA80 (an antioxidant manufactured by Sumitomo Chemical Co., Ltd.), 0.1 part by weight of Ultranox® 626 (an antioxidant manufactured by GE Specialty Chemicals, Inc.) and 0.3 part by weight of Sumilizer® TPM (an antioxidant manufactured by Sumitomo Chemical Co., Ltd.) were kneaded and granulated at 230° C. with a twin-screw extruder having a diameter of 40 mm to obtain a resin composition.

The resin composition prepared in the previous step was molded to produce a box-shaped molded article as shown in FIG. 1. The molded article had silver metallic appearance. It had faint black streak-like weld lines.

Then, a test piece having the outer sizes of 120 mm×120 mm×3 mm thick, shown in FIG. 2, was placed in an oven kept at 150° C. After heating the test piece for 150 hour, a ΔE value was measured to evaluate color change. The result is shown in Table 2.

### COMPARATIVE EXAMPLE 2

One hundred (100) parts by weight of an ethylene-propylene block copolymer (Sumitomo Noblen® AZ 864 manufactured by Sumitomo Chemical Co., Ltd.; MFR=30 g/10 min.) as a thermoplastic resin (A), 43 parts by weight of talc (MW HS-T manufactured by Hayashi-Kasei Co., Ltd.) as an inorganic filler (D), 16 parts by weight of an ethylene-butene copolymer (Sumitomo Excellen FX® CX5505 manufactured by Sumitomo Chemical Co., Ltd.; MFR=15 g/10 min.) as a thermoplastic elastomer (E), 1.7 parts by weight of an aluminum paste (manufactured by Toyo Aluminum Kabushiki-kaisha; average particle size: 10 μm) as a luster material (C), 0.32 part by weight of ADK STAB® ZS-90 (manufactured by ADEKA CORPORATION) as a metal deactivator and, as other additives, 0.1 part by weight of Sumilizer® GA80 (an antioxidant manufactured by Sumitomo Chemical Co., Ltd.), 0.1 part by weight of Ultranox® 626 (an antioxidant manufactured by GE Specialty Chemicals, Inc.) and 0.3 part by weight of Sumilizer® TPM (an antioxidant manufactured by Sumitomo Chemical Co., Ltd.) were kneaded and granulated at 230° C. with a twin-screw extruder having a diameter of 40 mm to obtain a resin composition.

The resin composition prepared in the previous step was molded to produce a box-shaped molded article as shown in FIG. 1. The molded article had silver metallic appearance. It had black streak-like weld lines.

Then, a test piece having the outer sizes of 120 mm×120 mm×3 mm thick, shown in FIG. 2, was placed in an oven kept at 150° C. After heating the test piece for 150 hour, a ΔE value was measured to evaluate color change. The result is shown in Table 3.

### COMPARATIVE EXAMPLE 3

One hundred (100) parts by weight of an ethylene-propylene block copolymer (Sumitomo Noblen® AZ 864 manufactured by Sumitomo Chemical Co., Ltd.; MFR=30 g/10 min.) as a thermoplastic resin (A), 21 parts by weight of talc (MW HS-T manufactured by Hayashi-Kasei Co., Ltd.) as an inorganic filler (D), 21 parts by weight of an ethylene-butene copolymer (Sumitomo Excellen FX® CX5505 manufactured by Sumitomo Chemical Co., Ltd.; MFR=15 g/10 min.) as a thermoplastic elastomer (E), 1.6 parts by weight of an aluminum paste (manufactured by Toyo Aluminum Kabushiki-kaisha; average particle size: 60 μm) as a luster material (C) and, as other additives, 0.1 part by weight of Sumilizer® GA80 (an antioxidant manufactured by Sumitomo Chemical Co., Ltd.), 0.1 part by weight of Ultranox® 626 (an antioxidant manufactured by GE Specialty Chemicals, Inc.) and 0.3 part by weight of Sumilizer® TPM (an antioxidant manufactured by Sumitomo Chemical Co., Ltd.) were kneaded and granulated at 230° C. with a twin-screw extruder having a diameter of 40 mm to obtain a resin composition.

The resin composition prepared in the previous step was molded to produce a box-shaped molded article as shown in FIG. 1. The molded article had silver metallic appearance. It had black streak-like weld lines.

Then, a test piece having the outer sizes of 120 mm×120 mm×3 mm thick, shown in FIG. 2, was placed in an oven kept at 150° C. After heating the test piece for 150 hour, a ΔE value was measured to evaluate color change. The result is shown in Table 4.

### COMPARATIVE EXAMPLE 4

One hundred (100) parts by weight of an ethylene-propylene block copolymer (Sumitomo Noblen® AZ 864 manufactured by Sumitomo Chemical Co., Ltd.; MFR=30 g/10 min.) as a thermoplastic resin (A), 43 parts by weight of talc (MW HS-T manufactured by Hayashi-Kasei Co., Ltd.) as an inorganic filler (D), 16 parts by weight of an ethylene-butene copolymer (Sumitomo Excellen FX® CX5505 manufactured by Sumitomo Chemical Co., Ltd.; MFR=15 g/10 min.) as a thermoplastic elastomer (E), 4.9 parts by weight of an aluminum paste (manufactured by Toyo Aluminum Kabushiki-kaisha; average particle size: 90 μm) as a luster material (C), 0.32 part by weight of ADK STAB® ZS-90 (manufactured by ADEKA CORPORATION) as a metal deactivator and, as other additives, 0.1 part by weight of Sumilizer® GA80 (an antioxidant manufactured by Sumitomo Chemical Co., Ltd.), 0.1 part by weight of Ultranox® 626 (an antioxidant manufactured by GE Specialty Chemicals, Inc.) and 0.3 part by weight of Sumilizer® TPM (an antioxidant manufactured by Sumitomo Chemical Co., Ltd.) were kneaded and granulated at 230° C. with a twin-screw extruder having a diameter of 40 mm to obtain a resin composition.

The resin composition prepared in the previous step was molded to produce a box-shaped molded article as shown in FIG. 1. The molded article had silver metallic appearance. It had black streak-like weld lines.

Then, a test piece having the outer sizes of 120 mm×120 mm×3 mm thick, shown in FIG. 2, was placed in an oven kept at 150° C. After heating the test piece for 150 hour, a ΔE value was measured to evaluate color change.

The result is shown in Table 5.

TABLE 1
Example 1

| | | |
|---|---|---|
| (A) | Polypropylene resin (wt. parts) | 100 |
| (B) | Glass fiber (wt. parts) | 43 |
| | Average fiber length (μm) | 300 |
| (C) | Luster material (wt. parts) | 4.9 |
| | Average particle size (μm) | 90 |

| | |
|---|---|
| Metal deactivator (wt. part) | 0.28 |
| Heat resistance test: ΔE | 0.1 |
| Weld appearance | A |

TABLE 2
Comparative Example 1

| | | |
|---|---|---|
| (A) | Polypropylene resin (wt. parts) | 100 |
| (D) | Talc (wt. parts) | 27 |
| (C) | Luster material (wt. parts) | 1.3 |
| | Average particle size (μm) | 10 |

| | |
|---|---|
| Metal deactivator (wt. part) | 0.25 |
| Heat resistance test: ΔE | 0.3 |
| Weld appearance | B |

TABLE 3
Comparative Example 2

| | | |
|---|---|---|
| (A) | Polypropylene resin (wt. parts) | 100 |
| (D) | Talc (wt. parts) | 43 |
| (E) | Thermoplastic elastomer (wt. parts) | 16 |
| (C) | Luster material (wt. parts) | 1.7 |
| | Average particle size (μm) | 10 |

| | |
|---|---|
| Metal deactivator (wt. part) | 0.32 |
| Heat resistance test: ΔE | 0.2 |
| Weld appearance | B |

TABLE 4
Comparative Example 3

| | | |
|---|---|---|
| (A) | Polypropylene resin (wt. parts) | 100 |
| (D) | Talc (wt. parts) | 21 |
| (E) | Thermoplastic elastomer (wt. parts) | 21 |
| (C) | Luster material (wt. parts) | 1.6 |
| | Average particle size (μm) | 60 |

| | |
|---|---|
| Heat resistance test: ΔE | 0.4 |
| Weld appearance | B |

TABLE 5
Comparative Example 4

| | | |
|---|---|---|
| (A) | Polypropylene resin (wt. parts) | 100 |
| (D) | Talc (wt. parts) | 43 |
| (E) | Thermoplastic elastomer (wt. parts) | 16 |
| (C) | Luster material (wt. parts) | 4.9 |
| | Average particle size (μm) | 90 |

| | |
|---|---|
| Metal deactivator (wt. part) | 0.32 |
| Heat resistance test: ΔE | 0.2 |
| Weld appearance | B |

The above results confirm that the molded article according to the present invention has good silver metallic appearance and few or no weld lines.

What is claimed is:

1. A resin composition comprising (A) 100 parts by weight of a thermoplastic resin, (B) 1 to 70 parts by weight of a fibrous material having an average fiber length of 0.1 to 20 mm, and (C) 0.1 to 10 parts by weight of a luster material.

2. The resin composition according to claim 1, wherein the luster material (C) has an average particle size of 90 to 180 μm.

3. The resin composition according to claim 1, wherein the luster material (C) comprises aluminum particles.

4. The resin composition according to claim 1, wherein the thermoplastic resin (A) is a polypropylene resin.

5. The resin composition according to claim 1, which further comprises 1 to 50 parts by weight of an inorganic filler (D).

6. The resin composition according to claim 1, which further comprises 1 to 50 parts by weight of a thermoplastic elastomer (E)
